SEQUENCE LISTING (18 sequences)

SEQ ID NO: 1 - protein, 359 aa, Percisivirga ulvanivorans
DTEKTPLEEKDVFNEDYIKTSMIKALEWQEAHPIFAIHPTDWTNGAYYTGVARAHHTTKNMMYMAALKNQAVANNWQPYTRLYHADDVAISYSYLYVAENEKRRNFSDLEPTKKFLDTHLYEDNAWKAGTNRSKEDKTILWWWCDALFMAPPVINLYAKQSEQPEYLDEMHKYYMETYNRLYDKEEKLFARDSRFVWDGDDEDKKEPNGEKVFWSRGNGWVIGGLALLLEDMPEDYKHRDFYVNLYKEMASRILEIQPEDGLWRTSLLSPESYDHGEVSGSAFHTFALAWGINKGLIDKKYTPAVKKAWKAMANCQHDDGRVGWVQNIGAFPEPASKDSYQNFGTGAFLLAGSEILKMR

SEQ ID NO: 2 - protein, 18 aa, Percisivirga ulvanivorans
MNKSILLLVTLLSLYSCT

SEQ ID NO: 3 - DNA, 1080 bp, Artificial Sequence
  nucleic acid encoding the peptide SEQ ID NO 1
gatactgaaaaaacaccattagaggagaaggatgtttttaatgaagattatataaaaacttctatgataaaagcactagagtggcaagaagcacaccctatttttgctatacatcctacagactggactaatggtgcatactatacaggtgttgcaagagcacatcatacgactaaaaacatgatgtatatggctgcgttaaaaaatcaagcagtggctaataattggcaaccatacacacgtttgtatcatgctgatgatgtcgctatttcatatagctatttgtatgtagctgaaaacgaaaaacgaaggaatttttcagatttagagcctacgaaaaagtttttagatacacatttgtatgaggataatgcttggaaagcaggaactaatagaagtaaagaagacaaaaccattttatggtggtggtgtgatgctttatttatggcaccacctgtaattaatttgtatgcaaaacagtcagagcaacctgagtatctagacgaaatgcacaaatattatatggaaacctataacagattgtatgataaagaagaaaagttatttgcaagagattcaagatttgtttgggacggtgatgatgaagacaaaaaagaaccaaatggtgaaaaagtattttggtctagaggaaatggatgggtaatcggcggtttagcattattgctagaggatatgccagaagactacaagcatagagatttctacgtgaacttgtataaagaaatggctagtagaatattagaaattcaaccagaagatggtttatggagaacaagtttgttaagtccagaatcttacgatcacggtgaggttagtggtagtgctttccatacttttgctttggcttggggaattaataaaggtttaatagataaaaaatatacacctgccgttaagaaagcgtggaaagctatggctaattgtcagcatgatgatggtcgtgtaggttgggtacaaaacataggtgcttttccagagccagcttctaaggatagttatcagaattttggaactggagcttttttgttagctggaagtgaaattctaaaaatgagataa

SEQ ID NO: 4 - DNA, 54 bp, Artificial Sequence
  nucleic acid encoding the signal peptide of SEQ ID NO 2
atgaataaatcaatcttattactggttactttattaagcctttatagttgtact

SEQ ID NO: 5 - DNA, 28 bp, Artificial Sequence
  TAIL PCR primer UL_133R
ctaggttgtaatgtgttaggtgcatccc

SEQ ID NO: 6 - DNA, 26 bp, Artificial Sequence
  TAIL PCR primer UL_194R
gtgaatcgcgcataacttcccacacc

SEQ ID NO: 7 - DNA, 24 bp, Artificial Sequence
  TAIL PCR primer UL_285R
cccgtgtgcttacctttggcctgc

SEQ ID NO: 8 - DNA, 25 bp, Artificial Sequence
  TAIL PCR primer UL_426F
gcagctggaagaaccgaggtctttc

SEQ ID NO: 9 - DNA, 26 bp, Artificial Sequence
  TAIL PCR primer UL_582F
ccggaaccagaacgaggaagagaatc

SEQ ID NO: 10 - DNA, 26 bp, Artificial Sequence
  TAIL PCR primer UL_643F
ggaggaagagcacaaatgagatgggc

SEQ ID NO: 11 - DNA, 35 bp, Artificial Sequence
  TAIL PCR primer AfterUL 1F
cacgtaatctgggtaggtttttatatcatgatacc

SEQ ID NO: 12 - DNA, 26 bp, Artificial Sequence
  TAIL PCR primer AfterUL 2F
gcttctgtaggtgtgtatcctaaccc

SEQ ID NO: 13 - DNA, 29 bp, Artificial Sequence
  TAIL PCR primer AfterUL 3F
gctggacgtgtgtcttctttgtattacgc

SEQ ID NO: 14 - DNA, 16 bp, Artificial Sequence
  TAIL PCR primer AD1
tgwgnagwancasaga

SEQ ID NO: 15 - DNA, 16 bp, Artificial Sequence
  TAIL PCR primer AD2
agwgnagwancawagg

SEQ ID NO: 16 - DNA, 16 bp, Artificial Sequence
  TAIL PCR primer AD3
wgtgnagwancanaga

SEQ ID NO: 17 - DNA, 15 bp, Artificial Sequence
  TAIL PCR primer AD4
ntcgastwtsgwgtt

SEQ ID NO: 18 - DNA, 16 bp, Artificial Sequence
  TAIL PCR primer AD5
ngtcgaswganawgaa

(19) United States
(12) Patent Application Publication
(10) Pub. No.: US 2014/0302561 A1
(43) Pub. Date: Oct. 9, 2014

(54) 3S-RHAMNOSE-GLUCURONYL HYDROLASE, METHOD OF PRODUCTION AND USES

(71) Applicants: Pi Collen, Roscoff (FR); William Helbert, Roscoff (FR); Yannick Lerat, Ploubazlanec (FR); Jean-François Sassi, Ploubazlanec (FR)

(72) Inventors: Pi Collen, Roscoff (FR); William Helbert, Roscoff (FR); Yannick Lerat, Ploubazlanec (FR); Jean-François Sassi, Ploubazlanec (FR)

(73) Assignees: UNIVERSITÉ PIERRE ET MARIE CURIE, Paris (FR); CENTRE NATIONAL DE LA RECHERCHE SCIENTIFIQUE- CNRS, Paris Cedex 16 (FR); CENTRE D'ÉTUDE ET DE VALORISATION DES ALGUES, Pleubian (FR)

(21) Appl. No.: 14/345,718

(22) PCT Filed: Sep. 14, 2012
(86) PCT No.: PCT/FR2012/052054

§ 371 Date: Jun. 4, 2014

(30) Foreign Application Priority Data
Sep. 19, 2011 (FR) 1158307

(51) Int. Cl. C12N 9/24; C12P 19/00

(52) U.S. Cl. CPC C12N 9/2402; C12P 19/00
USPC 435/72; 435/201; 536/23.2; 435/320.1; 435/252.33; 435/254.23; 435/254.21; 435/348

(57) ABSTRACT

The present invention relates in particular to proteins, coding nucleic acid sequences for same, vectors comprising said coding sequences, a method for producing said proteins, and an oligosaccharide hydrolysis method using same. In particular, the invention relates to the protein of sequence SEQ ID no. 1. The present invention can be applied to the recycling of bio-natural resources formed by organisms and microorganisms including ulvans, in particular green algae.

### TECHNICAL FIELD

The present invention relates in particular to proteins, to nucleic acid sequences encoding these proteins, to vectors comprising these coding sequences, to a method for producing these proteins, and also to an oligosaccharide hydrolysis method using these proteins.

The present invention find its application, in particular, to the exploitation of natural bioresources consisting of organisms and microorganisms comprising ulvans, in particular green algae. In particular, it can be applied in the laboratory, for the analysis of these ulvans and also in the food-processing industry, in the cosmetics field and in the field of medicaments and pharmaceutical formulations where ulvan degradation products can be exploited.

In the description below, the references between square brackets [ ] refer to the list of references presented at the end of the text.

### PRIOR ART

Green algae belonging to the genus Ulvales (Ulva sp. and Enteromorpha sp.) are present everywhere on earth and are very commonly encountered on coasts. These algae are frequently involved in algal blooms promoted by eutrophication of coastal waters, giving rise to “green tides”.

Until now, this undesirable biomass has been of very low added value and is used essentially as compost.

The complex anionic polysaccharides present in the ulva cell wall, ulvans possess unusual structures and represent a source of biopolymers of which the functionalities have so far received little attention.

Ulvans are made up of various disaccharide repeating units constructed with rhamnose units, glucuronic acid units, iduronic acid units, and xylose units, and sulfates.

The two main repeating units are called aldobiuronic acid, or ulvanobiuronic acids, or respectively A (A_{3S}) and B (B_{3S}), the formulae of which are the following:

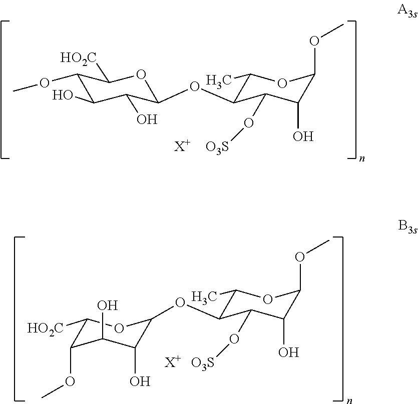

The A (A_{3S}) unit is beta-D-1,4-glucuronic acid (1→4) alpha-L-1,4-rhamnose 3-sulfate. The B (B_{3S}) unit is alpha-L-1,4-iduronic acid (1→4) alpha-L-1,4-rhamnose 3-sulfate.

The uronic acids are sometimes replaced with xylose residues which are sometimes sulfated at O-2.

Ulvans possess unique physicochemical properties that make them attractive candidates for new food-processing, pharmaceutical and cosmetic applications. Ulvans are composed of rare sugars such as rhamnose and iduronic acid. Rhamnose is an important compound of the surface antigens of numerous microorganisms that are recognized specifically by mammalian lectins. It is also used for the synthesis of flavorings. Iduronic acid is used for the synthesis of glycosaminoglycans (i.e. heparin).

In addition to the monomers, ulvans and oligo-ulvans have interesting biological properties. Indeed, studies have shown, for example, that oligo-ulvans have antitumor, antiviral (anti-flu) and anticoagulant activities. A non-exhaustive list of potential applications of ulvans has been proposed by M. Lahaye and A. Robic in the document Structure and functional properties of ulvan, a polysaccharide from green seaweeds. Biomacromolecules 2007, 8, 1765-1774 [1].

In this context, a better understanding of the structure of ulvans and the development of methods for fragmenting ulvans in oligomeric or monomeric form are of very great interest.

In this context, during a previous invention, the inventors have purified and cloned the gene of an ulvan lyase which specifically cleaves the glycosidic bond via a mechanism of elimination between the sulfated rhamnose and the uronic acids. The degradation products have been purified and the analyses of their structures have revealed that they systematically end, at their nonreducing end, with a disaccharide unit composed of glucuronyl acid and of a sulfated rhamnose. Thus, the ulvanlyase has made it possible to obtain specific oligosaccharides.

The oligosaccharides obtained are used, for example, in the cosmetics field, medical field, etc. However, the discovery of new enzymes for degradation or modification of ulvans and oligo-ulvans should make it possible to produce new series of oligosaccharides of well-calibrated molecular weights and structures, thus broadening the possibilities of application and of exploitation of ulvans.

Currently, through a lack of means for understanding them better and for degrading them efficiently, algae, in particular green algae, are essentially composted, without any industrial exploitation thereof. This is all the more deplorable since it is an abundant source which is sometimes troublesome in terms of pollution of our maritime coastlines. They are currently eliminated by composting.

There is therefore a real need to find novel means for degradation of ulvans and also oligosaccharides after degradation by ulvan lyases in order to be able to exploit this bioresource, resulting in particular from green algae, by producing “tailor-made” oligo-ulvan fragments with a view to cosmetic, food-processing and medical applications.

### SUMMARY OF THE INVENTION

The aim of the present invention is precisely to meet this need by providing proteins which very effectively hydrolyze oligosaccharides. Investigation of the modes of recognition of the enzymes of the present invention carried out by the inventors demonstrates their 3S-rhamnose-glucuronyl hydrolase activity.

A subject of the present invention is a protein of sequence SEQ ID No. °1 of the appended sequences listed, namely the peptide of sequence:

(SEQ ID No. 1)
DTEKTPLEEKDVFNEDYIKTSMIKALEWQEAHPIFAINPTDWTNGA
YYTGVARAHHTTKNMMYMAALKNQAVANNWQPYTRLYHADDVAISYSY
LYVAENEKRRNFSDLEPTKKFLDTHLYEDNAWKAGTNRSKEDKTILWWW
CDALFMAPPVINLYAKQSEQPEYLDEMHKYYMETYNRLYDKEEKLFARD
SRFVWDGDDEDKKEPNGEKVFWSRGNGWVIGGLALLLEDMPEDYKHR
DFYVNLYKEMASRILEIQPEDGLWRTSLLSPESYDHGEVSGSAFHTFALA
WGINKGLIDKKYTPAVKKAWKAMANCQHDDGRVGWVQNIGAFPEPASK
DSYQNFGTGAFLLAGSEILKMR

According to the invention the protein which is the subject of the invention is a 3S-rhamnose-glucuronyl hydrolase.

According to the invention, the protein which is the subject of the invention can also comprise, at its N-terminal end, a signal sequence or targeting sequence. This signal sequence may be one of the signal sequences known to those skilled in the art so that the protein, when it is synthesized in a host cell, is directed to an organelle or a particular region of the host cell. It may, for example, be a signal sequence found in the sites specialized in the prediction of signal peptides, for example http://www.cbs.dtu.dk/services/SignalP/ [2] or else http://bmbpcu36. leeds.ac.uk/prot_analysis/Signal.html [3]. It may, for example, be the sequence SEQ ID No. 2 of the appended sequence listing, namely the sequence MNKSILLLVTLLSLYSCT (SEQ ID No. 2). This signal sequence can be cleaved after synthesis of the protein or otherwise.

Advantageously, the inventors have noted that the signal sequences are not hindering and that the cleavage thereof is not necessary for the expression of protein, for example the protein is overexpressed without its signal peptide.

The present invention also relates to the nucleic acids encoding the protein of the present invention, in particular encoding the protein of sequence SEQ ID No. 1. The nucleic acid of the present invention may be any sequence encoding the peptide of sequence SEQ ID No. 1 taking account of the degeneracy of the genetic code. It may be, for example, a nucleic acid comprising or consisting of the sequence SEQ ID No. 3 of the appended sequence listing, namely the nucleic acid of sequence:

(SEQ ID No. 3)
GATACTGAAAAAACACCATTAGAGGAGAAGGATGTTTTTAATGAAGAT
TATATAAAAACTTCTATGATAAAAGCACTAGAGTGGCAAGAAGCACAC
CCTATTTTTGCTATACATCCTACAGACTGGACTAATGGTGCATACTATA
CAGGTGTTGCAAGAGCACATCATACGACTAAAAACATGATGTATATGG
CTGCGTTAAAAAATCAAGCAGTGGCTAATAATTGGCAACCATACACAC
GTTTGTATCATGCTGATGATGTCGCTATTTCATATAGCTATTTGTATGT
AGCTGAAAACGAAAAACGAAGGAATTTTTCAGATTTAGAGCCTACGAA
AAAGTTTTTAGATACACATTTGTATGAGGATAATGCTTGGAAAGCAGG
AACTAATAGAAGTAAAGAAGACAAAACCATTTTATGGTGGTGGTGTGA
TGCTTTATTTATGGCACCACCTGTAATTAATTTGTATGCAAAACAGTCA
GAGCAACCTGAGTATCTAGACGAAATGCACAAATATTATATGGAAACC
TATAACAGATTGTATGATAAAGAAGAAAAGTTATTTGCAAGAGATTCAA
GATTTGTTTGGGACGGTGATGATGAAGACAAAAAAGAACCAAATGGTG
AAAAAGTATTTTGGTCTAGAGGAAATGGATGGGTAATCGGCGGTTTAG
CATTATTGCTAGAGGATATGCCAGAAGACTACAAGCATAGAGATTTCT
ACGTGAACTTGTATAAAGAAATGGCTAGTAGAATATTAGAAATTCAACC
AGAAGATGGTTTATGGAGAACAAGTTTGTTAAGTCCAGAATCTTACGA
TCACGGTGAGGTTAGTGGTAGTGCTTTCCATACTTTTGCTTTGGCTTG
GGGAATTAATAAAGGTTTAATAGATAAAAAATATACACCTGCCGTTAAG
AAAGCGTGGAAAGCTATGGCTAATTGTCAGCATGATGATGGTCGTGTA
GGTTGGGTACAAAACATAGGTGCTTTTCCAGAGCCAGCTTCTAAGGAT
AGTTATCAGAATTTTGGAACTGGAGCTTTTTTGTTAGCTGGAAGTGAA
ATTCTAAAAATGAGATAA

According to the invention, the nucleic acid encoding the protein of the present invention may also comprise, at its 5′ end, the sequence SEQ ID No. 4 of the appended sequence listing, namely a nucleic acid of sequence

(SEQ ID No. 4)
ATGAATAAATCAATCTTATTACTGGTTACTTTATTAAGCCTTTATAGTTG
TACT.

In other words, the present invention also relates to an isolated nucleic acid as defined above.

The present invention also relates to a vector comprising a nucleic acid encoding the protein of the present invention, for example a nucleic acid of sequence SEQ ID No. 3 of the appended sequence listing.

The vector may be one of the vectors known to those skilled in the art for producing proteins by genetic recombination. It is generally chosen in particular according to the chosen host cell. The vector may be, for example, chosen from the vectors listed in the catalogue http://www.promega.com/vectors/mammalian_express_vectors.htm [4] or http://www.qiagen.com/pendantview/qiagenes.aspx?gaw=PROTQIAgenes0807&gkw=mammalian+expression [5], or else http://www.scbt.com/chap_exp_vectors.php?type=pCruzTM%20Expression%20Vectors [6]. It may, for example, be the expression vector described in document WO 83/004261 [7].

The nucleic acids of the present invention or the vectors of the present invention can be used in particular for producing proteins of the present invention by genetic recombination. Thus, the present invention also relates to a host cell comprising a nucleic acid sequence according to the invention or a vector according to the invention.

According to the invention, when the nucleic acid is in a host cell, it may or may not be isolated.

The host cell or cell host may be any host suitable for the production of the ulvan lyases of the present invention from the nucleic acids or the vectors of the invention. It may, for example, be E. coli, Pischia pastoris, Saccharomyces cerevisiae, insect cells, for example an insect cell-baculovirus system (for example SF9 insect cells used in a baculovirus expression system), or mammalian cells.

The host cell may also be the microorganism deposited under number I-4324 with the CNCM [French national collection of microorganism cultures] in France.

The present invention therefore also relates to a method for producing proteins of the present invention comprising the culturing of a host cell comprising a nucleic acid sequence according to the invention or a vector or the microorganism deposited under number I-4324 with the CNCM in France according to the invention.

This culturing is preferably carried out in a culture medium which allows the growth of the microorganism. It may be, for example, ZoBell liquid culture medium, as described in the document ZoBell, CE 1941 Studies on marine bacteria. I. The cultural requirements of heterotrophic aerobes, J Mar Res 4, 41-75 [8]. Culture conditions which can be used for implementing the present invention are also described in said document. The culture pH is preferably between 7 and 9, and is preferably pH 8. The culture temperature is preferably between 15 and 30° C., preferably 25° C. The culturing is preferably carried out with an NaCl concentration of from 20 to 30 g·l⁻¹, preferably of 25 g·l⁻¹.

This method for producing the proteins according to the invention using the microorganism deposited under number I-4324 with the CNCM in France or any other host cell transformed for a production by genetic recombination in accordance with the present invention, may also comprise a step of recovering the proteins according to the invention. This recovering or isolating step can be carried out by any means known to those skilled in the art. It may, for example, involve a technique chosen from electrophoresis, molecular sieving, ultracentrifugation, differential precipitation, for example with ammonium sulfate, by ultrafiltration, membrane or gel filtration, ion exchange, elution on hydroxyapatite, separation by hydrophobic interactions, or any other known means. An example of a method for isolating these 3S-rhamnose-glycuronyl hydrolases that can be used for implementing the present invention is described below.

The abovementioned microorganism or any other host cell transformed for a production by genetic recombination in accordance with the present invention may also be used directly for degrading oligosaccharides, in their natural environment or in culture. When a culture is involved, it may be a batchwise or continuous system. A culture reactor containing a culture medium suitable for the growth of the microorganism may, for example, be used.

The subject of the present invention is also an oligosaccharide hydrolysis method comprising a step of bringing the oligosaccharides into contact with a protein having a sequence of the invention, for example a protein of sequence SEQ ID No. 1, or with a host cell comprising a vector with a nucleic acid encoding the protein of the invention, for example a nucleic acid of sequence SEQ ID No. 3, under conditions which allow hydrolysis of the oligosaccharides.

In the present text, the term “oligosaccharides” is intended to mean oligomers made up of a number n of monosaccharides, i.e. monosaccharides via alpha- or beta-glycosidic bonding. They may be, for example, di-, tri-, or tetraoligosaccharides resulting from the degradation of ulvan by ulvan lyase which specifically cleaves the glycosidic bond via a mechanism of elimination between the sulfated rhamnose and the uronic acids. These degradation products may comprise, at their non-reducing end, a disaccharide unit composed of glucuronyl acid and of sulfated rhamnose in position 3. They may be, for example, oligosaccharides which have at least one glucuronyl acid bonded to a sulfated rhamnose, for example a glucuronyl acid bonded to a sulfated rhamnose which is itself bonded to a uronic acid.

For the enzymatic digestion, determination of the Michaelis-Menten constants (Km and Vmax) readily allows those skilled in the art to find the optimum conditions for concentrating the protein of the invention used and for concentrating the protein of the invention for the degradation of the protein of the invention in the medium in which it is present or the medium in which it has been placed. The pH may also preferably be between 7 and 8, preferably equal to 7.7. This is in fact the optimum pH range. The (optimum) temperature is preferably between 40° C. and 45° C. The optimum ionic strength may be equal to 100 mM NaCl with 100 mM Tris HCl or 200 mM NaCl.

The invention advantageously makes it possible to mobilize the very large resource of algae currently unexploited, in particular green algae. The invention also makes it possible to promote the biodegradation of algae, in particular of green algae, to produce unusual molecules, which are oligosaccharide fragments, for example also hydrocolloids for cosmetic, food-processing or medicament applications or pharmaceutical and parapharmaceutical formulations.

The oligosaccharide degradation products as defined above provide access to new products which may be food, cosmetic, pharmaceutical and parapharmaceutical active agents that can be used in the food-processing, cosmetics, pharmaceutical and parapharmaceutical fields. These new products may also be products which are not active but which exhibit a neutrality and/or a stability which is highly advantageous for use in each of these fields.

The use of the proteins of the invention also provides access to rare oligosaccharides that can be used as synthons in glycochemistry. The oligosaccharide degradation can provide access to iduronic acid (rare sugar) used for the synthesis of synthetic glycosaminoglycans.

The present invention also opens up new perspectives for use of these algae for applications in bioenergy and in chemistry. The production of oligosaccharide fragments can give basic molecules for the production of other molecules.

Other features and advantages will become further apparent to those skilled in the art upon reading the examples below, given by way of nonlimiting illustration, with reference to the appended figures.

### BRIEF DESCRIPTION OF FIGURES

FIG. 1 is a diagram of the genomic environment of the 3S-rhamnose-glucuronyl hydrolase gene of Percisivirga ulvanivorans. The dark part represents the gene encoding the protein of the invention.

FIG. 2 represents the protein sequence (SEQ ID No. 1) of the protein of the present invention with the signal peptide or sequence (SEQ ID No. 2) in bold.

FIG. 3 represents the SDS-PAGE gel of the protein of the invention, obtained after overexpression in E. coli and affinity column purification. The left-hand column represents the molecular weight markers.

FIG. 4 represents the results obtained from gel permeation chromatography experiments, representing the kinetics of modification of the oligo-ulvans by the protein of the invention. On this graph, the X-axis represents the retention time in minutes (min) and the Y-axis represents the refractive index.

FIG. 5 represents the results obtained from ion exchange chromatography experiments carried out with the major products of degradation of the ulvan by the ulvan lyase of P. ulvanivorans: (A) Δ-Rha3S; (B) Δ-Rha3S-GluA-Rha3S; (C) Δ-Rha3S-IduA-Rha3S; (D) Δ-Rha3S-Xyl-Rha3S with the protein of the present invention. On these graphs, the X-axis represents the elution time in minutes (min) and the Y-axis represents the conductimetry in microsiemens (μS).

FIG. 6 represents the schemes of enzymatic reactions catalyzed by the protein of the present invention. After cleavage of the glycosidic bond, the unsaturated monosaccharide produced spontaneously rearranges to give 4-deoxy-1-threo-5-hexosuloseuronic acid.

FIG. 7 represents the results for rate of cleavage of the glycosidic bond between the glucuronyl residue and the sulfated rhamnose as a function of the oligo-ulvan structure. On these graphs, the X-axis represents the time in minutes (min) and the Y-axis represents the absorbance (265 nm).

FIG. 8 is a diagram representing the subsite organization of the active site of the enzyme of the present invention, deduced from the experiments presented in FIG. 7. The + signs indicate the presence of potential cationic amino acids required for recognition of the sulfated rhamnose (subsite+1) and of the uronics (subsite+2).

FIG. 9 represents the results of proton NMR spectra of the trisaccharides obtained after incubation of the oligo-ulvan tetrasaccharides. A) mixture of glucuronic-rich oligo-ulvans (R3S-GlcA-R3S>R3S-IduA-R3S) or in B) of iduronic-rich oligo-ulvans (R3S-GlcA-R3S<R3S-IduA-R3S). C) trisaccharide of structure R3S-Xyl-R3S.

### EXAMPLES

### Example 1

### Identification and Production of the Hydrolase of the Invention

The genes contiguous to the ulvan lyase gene of the bacterium Persicivirga ulvanivorans called “01-PN-2010”, deposited with the CNCM under number I-4324, were sequenced by the “Tail PCR” method as described in Liu Y G, Mitsukawa N, Oosumi T and Whittier R F (1995) Efficient isolation and mapping of Arabidopsis thaliana T-DNA insert junctions by thermal asymmetric interlaced PCR. Plant J. 8: 457-463 [9].

A DNA fragment of 10934 base pairs was sequenced, revealing a group of 6 genes, the corresponding proteins of which exhibit homologies with glycoside hydrolase families classified in CAZY (http://www.cazy.org/) and a protein of unknown function.

The starting point for the sequencing by this method was the ulvan lyase gene. Three primers which pair (sense and antisense) with the known sequence were synthesized. Five different arbitrary primers were chosen from those proposed in the literature (table 1; Liu and Whittier 1995 [9]).

The primary TAIL PCR reactions were carried out in 20 μl reactions with 15 ng genomic DNA, 1× GoTaq PCR buffer, 1.5 mM MgCl₂, 0.2 mM of each dNTP, 0.2 μM of the first specific primer and a degenerate arbitrary primer (5 μM AD1 and AD2, 4 μM AD3, 2 μM AD4 and 3 μM AD5), and 1.25 U GoTaq (Promega).

The conditions for the secondary TAIL PCR reactions were identical to those of the primary reactions, except that 1 μl of a 1:50 dilution of the primary TAIL PCR reaction was used as strand of origin and a second specific primer was then used. For the tertiary TAIL PCR reaction 1 μl of a 1:50 dilution of the secondary TAIL PCR reaction was used with the third specific primer.

The amplification programs were adapted to each of the various TAIL PCR reactions according to the thermocyclers available in the laboratory (table 2). New primers specific for the gene of the GH105 protein, deduced from the TAIL-PCR experiments, were then synthesized in order to continue the sequencing of the gene.

TABLE 1
Primer used for identification
of the ulvan lyase gene

| | | | SEQ |
|---|---|---|---|
| | | | ID |
| Primers | Sequences (5' to 3') | Tₘ | No. |
| Primers | | | |
| specific | | | |
| for TAIL PCR | | | |
| UL_133R | CTAG GTT GTA ATG TGT TAG | 60 | 5 |
| | GTG CAT CCC | | |
| UL_194R | GTG AAT CGC GCA TAA CTT | 61 | 6 |
| | CCC ACA CC | | |
| UL_285R | CC CGT GTG CTT ACC TTT | 63 | 7 |
| | GGC CTG C | | |
| UL_426F | GC AGC TGG AAG AAC CGA | 61 | 8 |
| | GGT CTT TC | | |
| UL_582F | CCG GAA CCA GAA CGA GGA | 61 | 9 |
| | AGA GAA TC | | |
| UL_643F | GGA GGA AGA GCA CAA ATG | 61 | 10 |
| | AGA TGG GC | | |
| AfterUL 1F | CAC GTA ATC TGG GTA GGT | 61 | 11 |
| | TTT TAT ATC ATG ATA CC | | |
| AfterUL 2F | GCT TCT GTA GGT GTG TAT | 60 | 12 |
| | CCT AAC CC | | |
| AfterUL 3F | GCT GGA CGT GTG TCT TCT | 62 | 13 |
| | TTG TAT TAC GC | | |
| Degenerate | | | |
| arbitrary | | | |
| primers | | | |
| for TAIL PCR | | | |
| AD1 | TGW GNA GWA NCA SAG A | 38-43 | 14 |
| AD2 | AGW GNA GWA NCA WAG G | 38-43 | 15 |
| AD3 | WGT GNA GWA NCA NAG A | 38-43 | 16 |
| AD4 | NTC GAS TWT SGW GTT | 36-39 | 17 |
| AD5 | NGT CGA SWG ANA WGA A | 38-43 | 18 |

TABLE 2
Amplification programs

| | No. of | |
|---|---|---|
| Reaction | cycles | Temperature condition |
| Primary | 1 | 93° C., 2 min |
| | 5 | 94° C., 1 min; 62° C., 1 min; 72° C., 2 min |
| | 2 | 94° C. 1 min; increasing to 25° C. for 3 min; |
| | | 25° C., 3 min; |
| | | increasing to 72° C. for 3 min; |
| | | 72° C., 2 min |
| | 15 | 94° C., 30 sec; 65° C., 1 min; 72° C., 2 min; |
| | | 94° C., 30 sec; 65° C., 1 min; 72° C., 2 min; |
| | | 94° C., 30 sec; 45° C., 1 min; 72° C., 2 min |
| | 1 | 72° C., 7 min; 4° C., ∞ |
| Secondary | 1 | 93° C., 1 min |
| | 13 | 94° C., 30 sec; 62° C., 1 min; 72° C., 2 min; |
| | | 94° C., 30 sec; 62° C., 1 min; 72° C., 2 min; |
| | | 94° C., 30 sec; 45° C., 1 min; 72° C., 2 min |
| | 1 | 72° C., 7 min; 4° C., ∞ |
| Tertiary | 1 | 93° C., 1 min |
| | 20 | 94° C., 30 sec; 45° C., 1 min; 72° C., 2 min |
| | 1 | 72° C., 7 min; 4° C., ∞ |

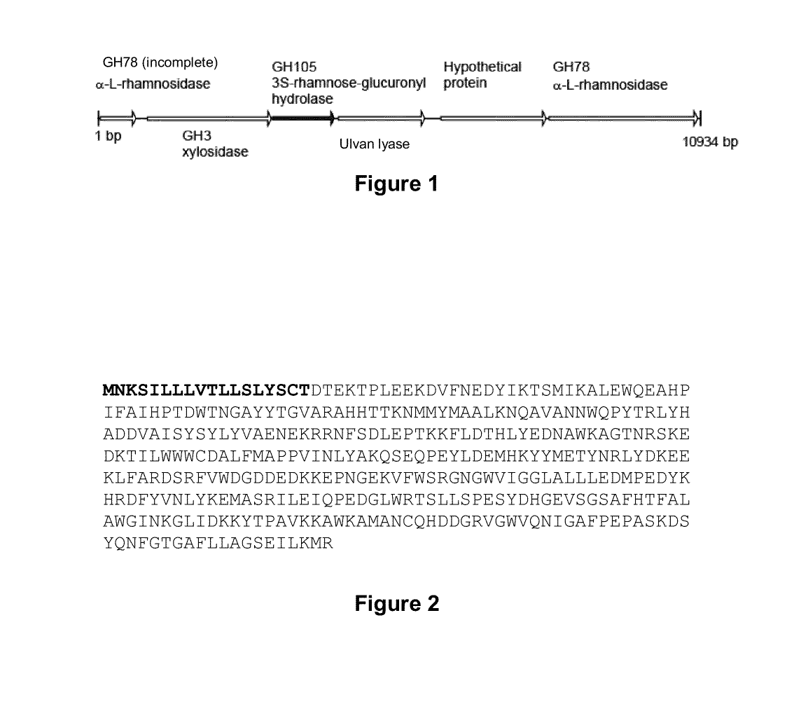

FIG. 1 represents the genomic environment of the ulvan lyase gene.

The gene encoding a protein belonging to the GH105 family has 1130 base pairs and the translation results in a protein having a molecular weight of 43.7 kD, composed of 377 amino acids, as represented in FIG. 2. The sequence analysis using the SignalP program (http://www.cbs.dtu.dk/services/SignalP/) made it possible to predict a signal peptide of 16 amino acids with a cleavage site between a serine (S16) and a cysteine (C17).

The complete gene without the signal peptide was cloned and introduced into the pFO4 expression vector according to the following protocol: primers pairing with the ends of the gene of the GH105 protein (excluding its signal peptide) and also having BamHI and EcoRI restriction sites at the 5′ and 3′ ends, respectively, of the gene were synthesized. These primers made it possible to amplify the gene according to standard PCR conditions with an annealing temperature of 50° C. and 30 cycles. The PCR products obtained were purified, digested with the appropriate restriction enzymes (BamHI/EcoRI) and were ligated into the pFO4 expression vector (modified pET15 (Novagen), Groisillier et al., 2010 Groisillier A, Hervé C, Jeudy A, Rebuffet E, Pluchon P F, Chevolot Y, Flament D, Geslin C, Morgado I M, Power D, Branno M, Moreau H, Michel G, Boyen C, Czjzek M 2010. MARINE-EXPRESS: taking advantage of high throughput cloning and expression strategies for the post-genomic analysis of marine organisms. Microb Cell Fact. 9, 45).

E. coli BL21 cells prepared in the laboratory according to the protocol of Cohen, S N, Chang A C Y, Hsu L (1972) Nonchromosomal antibiotic resistance in bacteria: genetic transformation of Escherichia coli by R-factor DNA. Proc. Natl. Acad. Sci. USA 69: 2110-2114 [11] were transformed with the plasmid by heat shock and then the expression of the gene was induced by the method of Korf U, Kohl T, vand der Zandt H Zahn R, Schleeger S Ueberle B, Wandschneider S Bechtel S, Schöler M, Ottleben H, Wiemann S and Poutska A 2005 Large scale protein expression for proteome research. Proteomics 2005, 5, 3571-3580, DOI 10.1002/pmic.200401195 [10] by incubating the transformed cells for 3 hours at 37° C. in a Luria-Bertani-based expression medium (10 g tryptone, 5 g yeast extract and 10 g NaCl per liter) with ampicillin and 0.5% glucose. An equal volume of cold Luria-Bertani medium with 0.6% lactose, 20 mM Hepes, pH 7.0 and 1 mM of isopropyl β-D-1-thiogalactopyranoside (IPTG) was added and the culture was incubated at 20° C. for 18 h.

The bacteria were recovered by centrifugation. The bacterial pellet was suspended in a 20 mM Tris-HCl buffer containing 500 mM NaCl and 5 mM imidazole at pH 7.4. The cells were lysed using a French press in a buffer of 20 mM Tris-HCl, 20 mM imidazole and 0.5M NaCl at pH 7.4. The cell debris were removed by centrifugation. The supernatant was injected onto a Ni Sepharose column loaded with 100 mM NiSO₄ (GE Healthcare). After washing, the proteins retained were eluted with a liner gradient of imidazole from 20 mM to 500 mM. The active fractions were collected and purified on a superdex 75 column (1.6×60 cm; GE Healthcare) equilibrated with 20 mM Tris-HCl, pH 8.0, with 200 mM NaCl then samples were prepared by mixing 10 μl of active fraction active with 10 μl of loading buffer containing SDS and were heated at 95° C. for 5 min before migration in an electrophoresis gel. The gel was a precast SDS-12% polyacrylamide gel (Mini Protean TGX, Biorad), and the migration was carried out at 200 V and 20 mA for 2 h. The amount of protein loaded per well was between 1 and 10 μg. The molecular weight markers were 5 μl precision plus protein standards (Biorad). The gel was developed using coomassie staining.

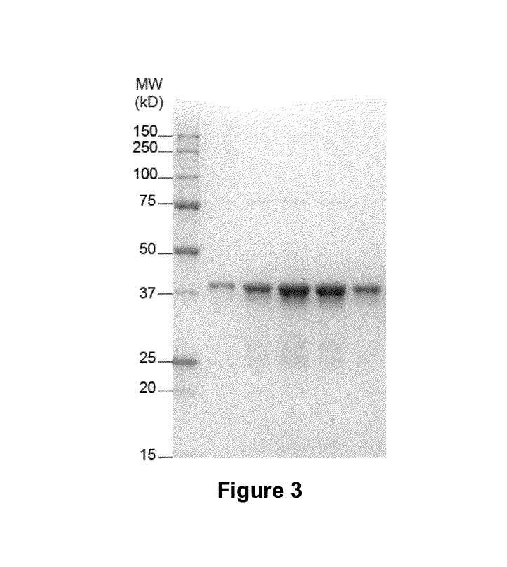

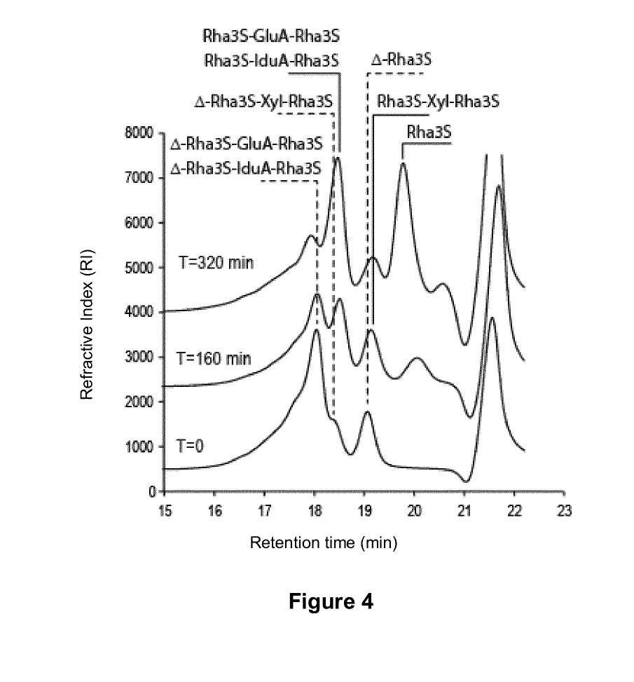

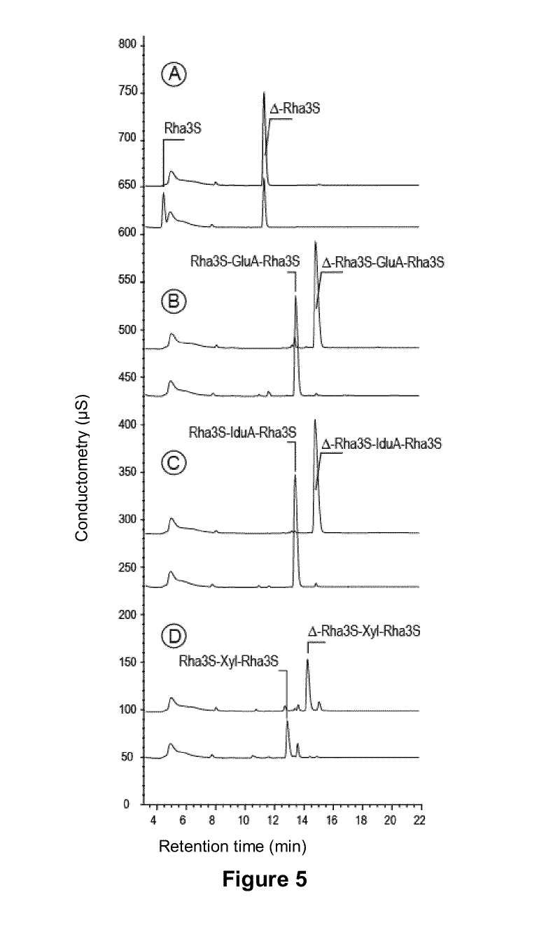

FIG. 3 represents the gel obtained after migration. The protein was strongly expressed, as demonstrated by the electrophoresis gel. Indeed, the strongest band corresponds to a protein, the molecular weight of 42 kD of which corresponds to that expected.

### Example 2

### Demonstration of the Enzymatic Activity of the Protein of the Invention

Enzymes described in the literature belonging to the GH105 family are known to cleave the bond between the galacturonyl acid and the neutral rhamnose of the oligosaccharides produced after degradation of rhamnogalacturonan by the rhamnogalacturonan lyases. Galactose is the C4 epimer of glucose, consequently, the formation of the double bond between the C4 and C5 of galactose and of glucose results in a uronic acid having the same chemical structure: galacturonyl acid is synonymous with glucuronyl acid. An oligosaccharide production was carried out by degradation of polygalacturonan at 1 g/l in 100 mM Tris-HCl at pH 7.7, at 30° C., with a rhamnogalacturonan lyase (novozymes) at a final concentration of 0.2 μg/ml. The degradation was followed by an increase in absorbance at 235 nm.

These oligosaccharides were thus incubated under these same conditions with the protein of the invention at the usual final degradation concentration of 0.05 μg/ml and no decrease in the absorbance at 235 nm was observed.

Surprisingly, the protein of the invention did not appear to eliminate the galacturonyl (=glucuronyl) residue like the GH105 proteins studied and known in the prior art.

An oligo-ulvan production was carried out by degradation of the ulvan using the “01-PN-2010” ulvan lyase until completion, followed by a step of ultrafiltration on a 5000 kD membrane in order to remove the resistant fraction. The mixture obtained had a concentration of 12.5 mM of oligosaccharides, predominantly di- and tetrasaccharides, but the presence of hexa-, octa- and decasaccharides was also observed.

The oligo-ulvan mixture obtained after degradation of the ulvan using “01-PN-2010” ulvan lyase was incubated with the protein of the invention at a final concentration of 0.05 μg/ml at ambient temperature (20° C.).

The oligo-ulvan degradation kinetics were monitored by gel permeation on a Superdex 200 column coupled in series with a peptide HR column (GE Healthcare). The elution was carried out in 50 mM ammonium carbonate at a flow rate of 0.5 ml min⁻¹ on an Ultimate 3000 HPLC system (Dionex) equipped with a UV detector (Dionex) at 235 nm and an RI refractometer (Wyatt). The injected volume was 100 μl.

Before incubation with the GH105 protein of the invention, the peaks were assigned to the oligo-ulvans which have the property of being detected both by UV (265 nm) and according to their refractive index.

During the degradation, the absorbance at 235 nm decreases, indicating that the glucuronyl acid of the non-reducing end is removed. The peaks detected by their refractive index were reduced and then disappeared to the benefit of new signals as represented.

The systematic degradation of the oligo-ulvans by the 3S-rhamnose-glucuronyl hydrolase was confirmed by high performance ion exchange chromatography (HPAEC) on a Dionex ICS 3000 chromatography apparatus equipped with a 20 μl injection loop, with an AS100XR automated injection system (Thermo Separation Products) and with an AS11 ion exchange column (4 mm×250 mm, Dionex IonPac) combined with an AG11 guard precolumn (4 mm×50 mm, Dionex IonPac). The system was operated in conductivity mode with an ED40 detector (Dionex) and an ASRS ultra-4mm suppressor (Dionex) at 300 mA. The mobile phases were ultrapure water (solution A) and 290 mM NaOH (solution B). The elution was carried out at a flow rate of 0.5 ml min⁻¹ and the gradient used was 0 min: 3% Sol. B; 1.5 min: 1% Sol. B; 4.1 min: 5% Sol. B; 6.5 min: 10% Sol. B; 10.0 min: 18% Sol. B; 26 min: 22% Sol. B; 28 min; 40% Sol. B; 30 min: 100% Sol. B; 30.1 min: 3% Sol. B; 37 min: 3% Sol. B. The data acquisition and analysis were carried out with the Chromeleon-peak Net software (Dionex).

The four major oligo-ulvans were purified and then incubated with the protein of the invention. The reaction medium was composed of 100 mM Tris HCl, pH 7.7, 100 mM NaCl with 125 μM of purified oligosaccharides and a final protein concentration of 0.05 μg/ml. The reaction was carried out at 30° C. and monitored with a spectrophotometer at 235 nm. The molecular weight of these four oligosaccharides was reduced and the molecules lost their properties of absorbing the UV at 265 nm.

The oligosaccharides obtained after incubation of the protein of the invention were analyzed by proton ¹H NMR and carbon NMR. The spectra were obtained with a Bruker Avance DRX 500 spectrophotometer (NMR Department of the Université de Bretagne Occidentale [University of West Brittany]) at 20° C. Before the analysis, the samples were transferred into D₂O (99.97 atom % D₂O).

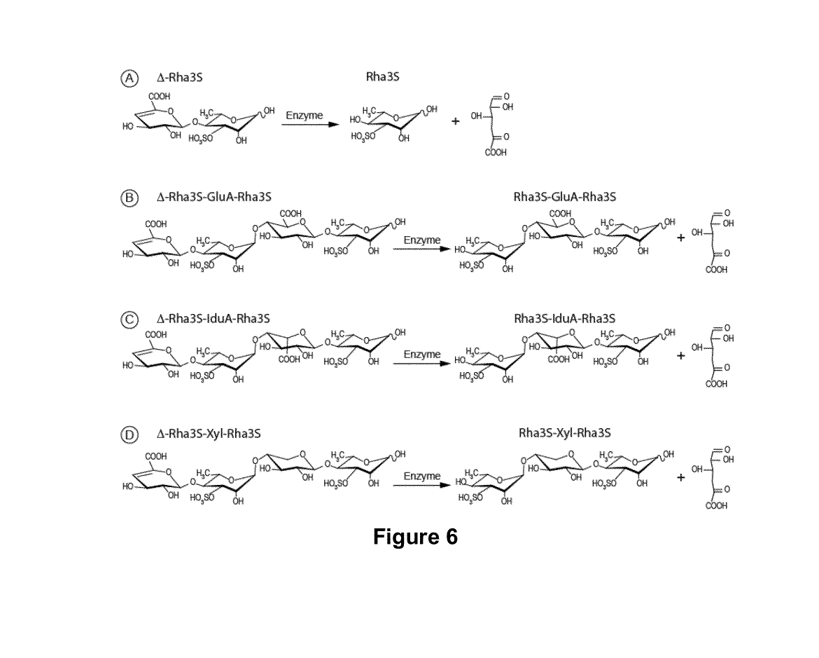

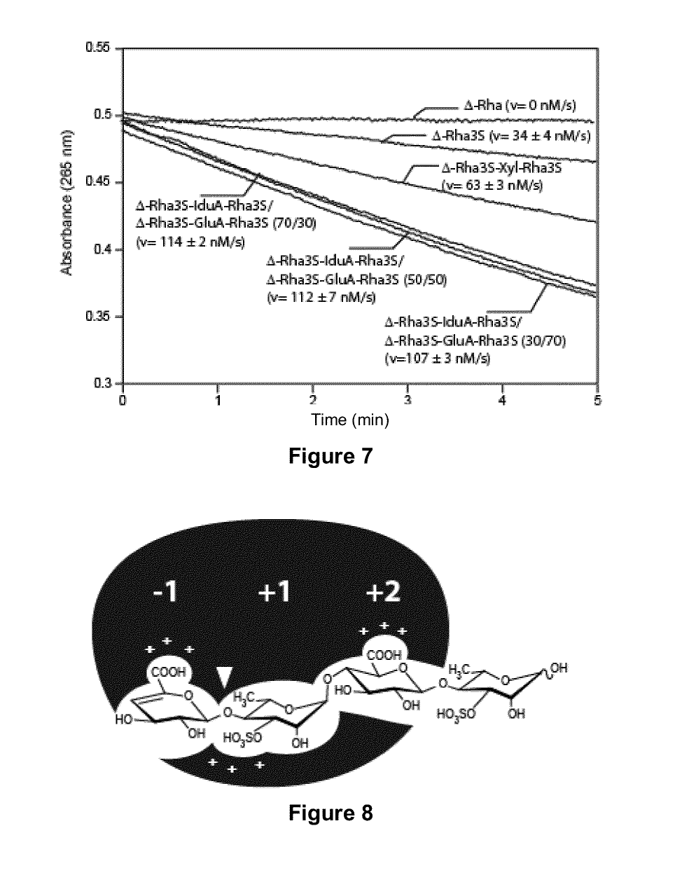

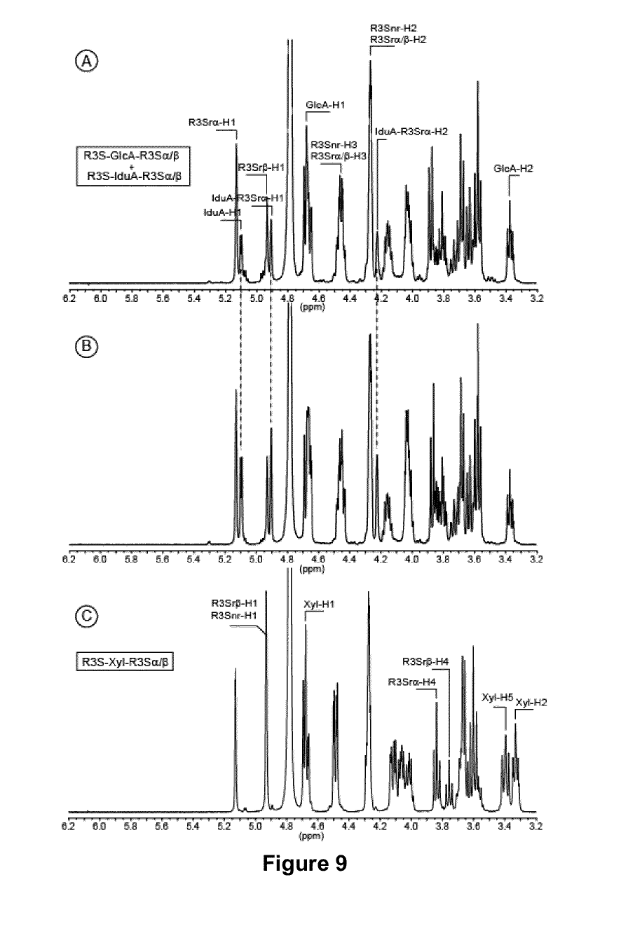

The spectra clearly indicate the disappearance of the glucuronyl acid unit and that the oligosaccharides are ended, at their non-reducing end, with a sulfated rhamnose. The structure of the oligosaccharides demonstrates that the protein of the invention is a 3S-rhamnose-glucuronyl hydrolase which catalyzes the hydrolysis of the glycosidic bond between the glucuronyl acid and the sulfated rhamnose. These enzymes catalyze in particular the reactions presented in FIG. 6.

The study of the kinetics of degradation of the purified oligosaccharides by the protein of the invention was carried out in a reaction medium composed of 100 mM NaCl, 100 mM Tris HCl (pH 7.7) and ulvan oligosaccharides (dp 2-8) at 30° C. in a 1 ml quartz cuvette. The maximum oligo-ulvan concentration used corresponds to an absorbance of 0.5 at 235 nm. 10 μl of pure GH105 (19 μg/ml) was added to a reaction medium. The ulvan oligosaccharide degradation (or rather the disappearance of the non-reducing glucuronyl acid) was monitored through the decrease in absorbance at 235 nm for 5 min.

This made it possible to refine the recognition modes. The inventors demonstrated that the tetrasaccharides having the structures Δ-Rha3S-GluA-Rha3S and Δ-Rha3S-IduA-Rha3S are degraded more rapidly. A decrease in degradation rate observed for the oligo-ulvan Δ-Rha3S-Xyl-Rha3S indicates that the presence of the uronic function at +2 of the active site is important. This observation suggests that the active site is organized into 3 subsites, which is confirmed by the rate of degradation of disaccharide Δ-Rha3S. The presence of the sulfate on the rhamnose is essential for obtaining the substrate recognition since the Δ-Rha disaccharide obtained with rhamnogalacturonan is not degraded.

### REFERENCE LIST

[1] Marc Lahaye and Audrey Robic, Structure and functional properties of ulvan, a polysaccharide from green seaweeds. Biomacromolecules 2007, Vol. 8, 1765-1774.
[2] http://www.cbs.dtu.dk/services/Signal P/
[3] http://bmbpcu36.leeds.ac.uk/prot_analysis/Signal.htmL
[4] http://www.promega.com/vectors/mammalian_express_vectors.htm
[5] http://www.qiagen.com/pendantview/qiagenes.aspx?gaw=PROTQIAgenes0807&gkw=mammalian+expression
[6] http://www.scbt.com/chap_exp_vectors.php?type=pCruzTM%20Expression%20Vectors
[7] WO 83/004261
[8] ZoBell, C E 1941 Studies on marine bacteria. I. The cultural requirements of heterotrophic aerobes, J Mar Res 4, 41-75
[9] Liu Y G, Mitsukawa N, Oosumi T and Whittier R F (1995) Efficient isolation and mapping of Arabidopsis thaliana T-DNA insert junctions by thermal asymmetric interlaced PCR. Plant J. 8: 457-463
[10] Korf U, Kohl T, vand der Zandt H Zahn R, Schleeger S Ueberle B, Wandschneider S Bechtel S, Schöler M, Ottleben H, Wiemann S and Poutska A 2005 Large scale protein expression for proteome research. Proteomics 2005, 5, 3571-3580, DOI 10.1002/pmic.200401195
[11] Cohen, S N, Chang A C Y, Hsu L (1972) Nonchromosomal antibiotic resistance in bacteria: genetic transformation of Escherichia coli by R-factor DNA. Proc. Natl. Acad. Sci. USA 69: 2110-2114
[12] Groisillier A, Hervé C, Jeudy A, Rebuffet E, Pluchon P F, Chevolot Y, Flament D, Geslin C, Morgado I M, Power D, Branno M, Moreau H, Michel G, Boyen C, Czjzek M 2010. MARINE-EXPRESS: taking advantage of high throughput cloning and expression strategies for the post-genomic analysis of marine organisms. Microb Cell Fact. 9, 45.

1. A protein of sequence SEQ ID No. 1 of the appended sequence listing.

2. The protein as claimed in claim 1, also comprising, at its N-terminal end, a signal sequence.

3. The protein as claimed in claim 2, wherein the signal sequence is the sequence SEQ ID No. 2 of the appended sequence listing.

4. An isolated nucleic acid of sequence SEQ ID No. 3 of the appended sequence listing.

5. The isolated nucleic acid as claimed in claim 4, also comprising at its 5′ end, the sequence SEQ ID No. 4 of the appended sequence listing.

6. A vector comprising an isolated nucleic acid as claimed in claim 4.

7. A host cell comprising an isolated nucleic acid sequence as claimed in claim 4.

8. A method for producing a protein as claimed in claim 1 by genetic recombination using an isolated nucleic acid of sequence SEQ ID No. 3 of the appended sequence listing.

9. An oligosaccharide hydrolysis method comprising a step of bringing the oligosaccharides into contact with a protein as claimed in claim 1 or with a host cell comprising an isolated nucleic acid of sequence SEQ ID No. 3 of the appended sequence listing under conditions which allow hydrolysis of the oligosaccharides.

10. A vector comprising an isolated nucleic acid as claimed in claim 5.

11. A host cell comprising an isolated nucleic acid sequence as claimed in claim 5.

12. A host cell comprising a vector as claimed in claim 6.

13. A host cell comprising a vector as claimed in claim 10.

14. A method for producing a protein as claimed in claim 1 by genetic recombination using an isolated nucleic acid of sequence SEQ ID No. 3 of the appended sequence listing comprising at its 5′ end, the sequence SEQ ID No. 4 of the appended sequence listing.

15. A method for producing a protein as claimed in claim 1 by genetic recombination using a vector comprising an isolated nucleic acid of sequence SEQ ID No. 3 of the appended sequence listing.

16. An oligosaccharide hydrolysis method comprising a step of bringing the oligosaccharides into contact with a protein as claimed in claim 1 or with a host cell comprising an isolated nucleic acid of sequence SEQ ID No. 3 of the appended sequence listing also comprising at its 5′ end, the sequence SEQ ID No. 4 of the appended sequence listing, under conditions which allow hydrolysis of the oligosaccharides.

17. An oligosaccharide hydrolysis method comprising a step of bringing the oligosaccharides into contact with a host cell comprising an isolated nucleic acid of sequence SEQ ID No. 3 of the appended sequence listing, under conditions which allow hydrolysis of the oligosaccharides.

18. An oligosaccharide hydrolysis method comprising a step of bringing the oligosaccharides into contact with a host cell comprising an isolated nucleic acid f sequence SEQ ID No. 3 of the appended sequence listing also comprising at its 5′ end, the sequence SEQ ID No. 4 of the appended sequence listing, under conditions which allow hydrolysis of the oligosaccharides.

19. An oligosaccharide hydrolysis method comprising a step of bringing the oligosaccharides into contact with a host cell comprising A vector comprising an isolated nucleic acid of sequence SEQ ID No. 3 of the appended sequence listing, under conditions which allow hydrolysis of the oligosaccharides.

20. An oligosaccharide hydrolysis method comprising a step of bringing the oligosaccharides into contact with a host cell comprising a vector comprising an isolated nucleic acid of sequence SEQ ID No. 3 of the appended sequence listing also comprising at its 5′ end, the sequence SEQ ID No. 4 of the appended sequence listing, under conditions which allow hydrolysis of the oligosaccharides.
